(19) United States
(12) Patent Application Publication
(10) Pub. No.: US 2007/0066499 A1
(43) Pub. Date: Mar. 22, 2007

(54) Self-supporting aerosol cleansing composition

(75) Inventors: Craig Stephen Slavtcheff, Guilford, CT (US); Michael A. Brennan, Sandy Hook, CT (US); Sandra Dawn Meraviglia, Milford, CT (US); Jessica Weiss Goldberg, Fairfield, CT (US); Michael Massaro, Monroe, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/229,751

(22) Filed: Sep. 19, 2005

(51) Int. Cl. A61K 8/00

(52) U.S. Cl. 510/130

(57) ABSTRACT

An aerosol cleansing composition that is self-supporting after dispensing is described whose volume does not noticeably shrink with time. The inventive composition can be used in place of toilet bars when it is inconvenient to use such bars and provides the user with substantial lather and optionally warming agents, skin conditioners and/or skin active agents. A method for cleansing the skin without rinsing with water is also disclosed where the inventive aerosol cleansing composition is applied to the skin and wiped off.

### BACKGROUND OF THE INVENTION

1. Field of the Invention

The present invention relates to detergent compositions suitable for topical application for cleansing the human body, such as the skin and hair. In particular, it relates to a self-supporting aerosol cleansing composition.

2. The Related Art

U.S. Pat. No. 3,705,669 to Cox et al. Issued on Dec. 12, 1972 discloses a pressurized dispensing container containing a composition comprising a resin for forming a cohesive body of plastic foam, a surfactant for providing a surface of controlled tackiness on the foam and a propellant in which the resin and surfactant are soluble for propelling the composition from the container, and for subsequent expansion to forming the foam.

U.S. Pat. No. 3,912,665 to Spitzer et al. Issued on Oct. 14, 1975 discloses foam structures such as applicator pads for cleaning and other purposes, that are formed from emulsified propellant compositions including a synthetic polymer in solution in a low boiling propellant and dispersed or emulsified in an organic liquid that serves as the continuous phase.

U.S. Pat. No. 5,830,438 to Dupuis Issued on Nov. 3, 1998 discloses a composition used to prepare a hair-care foam comprising at least one cationic polymer and at least one amphoteric polymer possessing effective foaming properties and yielding a foam having excellent cosmetic properties.

U.S. Pat. No. 6,113,923 Le Bourhis Issued on Sep. 5, 2000 discloses a cosmetic composition comprising a pressurized aerosol composition spontaneously forming a foam on contact with an application surface, in particular keratinous material such as hair. The aerosol composition includes in one embodiment: (i) an aqueous vehicle containing at least one foaming anionic polymer and/or at least one foaming cationic polymer; and (ii) a mixture of propellants containing dimethyl ether and at least one other propellant selected from volatile alkanes and volatile haloalkanes.

U.S. Pat. No. 6,121,210 Taylor Issued on Sep. 19, 2000 discloses a silicone oil-based foamable lubricant capable of forming a stable foam when extruded through the valve of an aerosol canister or other spray container.

Surprisingly, a stable, self-supporting aerosol cleansing composition that can be conveniently dispensed in situations where the use of a toilet bar would be impractical or inconvenient was discovered. The inventive composition may be advantageously applied to the skin and wiped or washed off. Furthermore the inventive cleansing composition surprisingly forms a dense self-supporting mass on dispensing from an aerosol dispenser.

### SUMMARY OF THE INVENTION

In one aspect the present invention is an aerosol cleansing composition that is self-supporting after dispensing including but not limited to:

a. surfactant(s) with a Krafft point greater than 27 C in a total concentration range of about 20 to 60% by wt.;

b. optionally surfactant(s) with a Krafft point less than or equal to 27 C in a total concentration range of up to about 25% by wt.;

c. non-propellant solubilizing agent(s) in a total concentration range of about 20 to 80% by wt.;

d. propellant(s) in a total concentration range of about 2 to 20% by wt.; and

e. wherein the dispensed composition retains about 90% of its dispensed volume after 24 hours (at approx. 23 C, 760 torr or 101 Kpa, and 70% RH).

In another aspect of the invention is a packaged cleansing product including but not limited to:

a. a container having a valve;

b. an aerosol cleansing composition that is self-supporting after dispensing including: surfactant(s) with a Krafft point greater than 27 C in a total concentration range of about 20 to 60% by wt.; optionally surfactant(s) with a Krafft point less than or equal to 27 C in a total concentration range of up to about 25% by wt.; non-propellant solubilizing agent(s) in a total concentration range of about 20 to 80% by wt.; propellant(s) in a total concentration range of about 2 to 20% by wt.; and wherein the dispensed composition retains about 90% of its dispensed volume after 24 hours (at approx. 23 C, approx. 760 torr or 101 Kpa, and approx. 70% RH); and

c. wherein the valve is capable of releasing the cleansing composition when the internal pressure exerted by the cleansing composition is greater than the outside air pressure.

In a further aspect of the invention is a method for cleansing the skin without rinsing with water, including but not limited to the steps of applying the inventive self-supporting aerosol cleansing composition to the skin; distributing the applied composition to the area of skin desired; and wiping off the applied composition.

### DETAILED DESCRIPTION OF THE INVENTION

In one aspect the present invention is an aerosol cleansing composition that is self-supporting after dispensing including but not limited to:

a. surfactant(s) with a Krafft point greater than 27 C in a total concentration range of about 20 to 60% by wt.; preferably at least about 20, 25 or 30% by wt. and less than about 55, 50 or 45% by wt.;

b. optionally surfactant(s) with a Krafft point less than or equal to 27 C in a total concentration range of up to about 25% by wt.; preferably at least about 5, 7 or 10% by wt. and less than about 23, 20 or 18% by wt.;

c. non-propellant solubilizing agent(s) in a total concentration range of about 20 to 80% by wt.; preferably at least about 25, 30 or 35% by wt. and less than about 60, 55 or 50% by wt.;

d. propellant(s) in a total concentration range of about 2 to 20% by wt.; preferably at least about 1, 2 or 3% by wt. and less than about 10, 8 or 6% by wt., and

e. wherein the dispensed composition retains about 90% of its dispensed volume after 24 hours (at approx. 23 C, 760 torr or 101 Kpa, and 70% RH); preferably retaining at least about 95, 96 or 97% of its dispensed volume 5 minutes after dispensing.

Advantageously the inventive composition further includes a lather coefficient of at least 4 (at approx. 23 C, 760 torr or 101 Kpa, and 70% RH). preferably 5, and most preferably 6.

In a preferred embodiment, the inventive composition further includes one or more structurant(s) in a total concentration of at least 0.1% by wt.; preferably at least about 10, 20 or 25% by wt. and less than about 50, 40 or 30% by wt. Advantageously the ratio of total structurants to total surfactants is in the range of about 0.2 to 5; preferably where the ratio is at least about 0.5, 0.75 or 1.0 and less than about 1.1, 1.3 or 1.5.

Advantageously the inventive composition further includes one or more skin conditioning agents (also known as emollients) in the total concentration range of about 0.1 to 5% by wt.; preferably at least about 0.1, 0.3 or 0.5% by wt. and less than about 3, 2 or 1% by wt.

Propellants are an essential part of the inventive composition and are preferably selected from hydrocarbons, dimethyl ether, carbon dioxide, nitrous oxide, compressed air, nitrogen, fluorohydrocarbons, or blends thereof.

Preferably the specific gravity of the cleansing composition measured 5 seconds after dispensing is at least about 0.25 (at approx. 23 C, 760 torr or 101 Kpa, and 70% RH). More preferably the specific gravity is at least about 0.3, 0.5 or 0.7.

In a preferred embodiment, the soap content is less than about 40% by wt.; preferably less than about 30, 20, 10, 5, 4 or 3% by wt.

Advantageously the composition includes one or more skin active agent(s) in the total concentration range of about 0.02 to 10% by wt.; preferably at least about 0.05, 0.1, 0.5, 1 or 2% by wt. and less than about 10, 8 or 5% by wt. More preferably the skin active agent(s) are selected from antibacterial agent(s), anti-aging agent(s), anti-acne agent(s) or skin firming agent(s) or a blend thereof.

In a further preferred embodiment, the inventive composition further includes less than about 90% by wt. of water, and advantageously less than about 75, 60, 50, 40, 30, 20, 15, 10, 5, 3, 1 or 0.5% by wt. of water. In a preferred embodiment the composition is substantially anhydrous wherein it contains less than 15, 10, 5, 3, 2, 1, or 0.5.% by wt. of water and preferably the composition includes an effective concentration of heat generating compound(s) sufficient for generating an increase in temperature of the composition with use.

In another aspect of the invention is a packaged cleansing product including but not limited to:

a. a container having a valve;

b. a self-supporting aerosol composition including: surfactant(s) with a Krafft point greater than 27 C in a total concentration range of about 20 to 60% by wt.; optionally surfactant(s) with a Krafft point less than or equal to 27 C in a total concentration range of up to about 25% by wt.; non-propellant solubilizing agent(s) in a total concentration range of about 20 to 80% by wt.; propellant(s) in a total concentration range of about 2 to 20% by wt.; and wherein the dispensed composition retains about 90% of its dispensed volume after 24 hours (at approx. 23 C, approx. 760 torr or 101 Kpa, and approx. 70% RH); and

c. wherein the valve is capable of releasing the cleansing composition when the internal pressure exerted by the cleansing composition is greater than the outside air pressure.

In a further aspect of the invention is a method for cleansing the skin without rinsing with water, including but not limited to the steps of applying the inventive self-supporting aerosol cleansing composition to the skin; distributing the applied composition to the area of skin desired; and wiping off the applied composition.

Surfactants:

Surfactants are an essential component of the inventive cleansing composition. They are compounds that have hydrophobic and hydrophilic portions that act to reduce the surface tension of the aqueous solutions they are dissolved in. Useful surfactants include soap(s), and non-soap anionic, nonionic, amphoteric, and cationic surfactants, and blends thereof. Total surfactant(s) concentration will preferably range from about 20 to 60% by weight of the cleansing composition prior to dispensing. Preferably, this component is present from about 30% to 45% in the foaming cleansing composition prior to dispensing.

Anionic Surfactants:

The cleansing composition of the present invention may contain one or more anionic soap and non-soap surfactants. When used, the total anionic surfactants may preferably be present from about 10, 15 or 20% by wt. up to about 30, 35 or 40% by wt.

The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M

wherein R is an alkyl or alkenyl having 8 to 18 carbons, preferably 12 to 18 carbons, n has an average value of greater than 1.0, preferably greater than 3; and M is a solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:
R⁴O₂CCH₂CH(SO₃M)CO₂M; and

amide-MEA sulfosuccinates of the formula;
R⁴CONHCH₂CH₂O₂CCH₂CH(SO₃M)CO₂M

wherein R⁴ ranges from C₈-C₂₂ alkyl and M is a solubilizing cation.

Sarcosinates are generally indicated by the formula:
R¹CON(CH₃)CH₂CO₂M,

wherein R¹ ranges from C₈-C₂₀ alkyl and M is a solubilizing cation.

Taurates are generally identified by formula:
R²CONR³CH₂CH₂SO₃M

wherein R² ranges from C₈-C₂₀ alkyl, R³ ranges from C₁-C₄ alkyl and M is a solubilizing cation.

The inventive cleansing composition may contain C₈-C₁₈ acyl isethionates. These esters are prepared by reaction between alkali metal isethionate with mixed aliphatic fatty acids having from 6 to 18 carbon atoms and an iodine value of less than 20. Preferably at least 75% of the mixed fatty acids have from 12 to 18 carbon atoms and up to 25% have from 6 to 10 carbon atoms.

The acyl isethionate may be an alkoxylated isethionate such as is described in llardi et al., U.S. Pat. No. 5,393,466, titled “Fatty Acid Esters of Polyalkoxylated isethonic acid; issued Feb. 28, 1995; hereby incorporated by reference. This compound has the general formula:
RC—O(O)—C(X)H—C(Y)H₂—(OCH—CH₂)ₘ—SO₃M⁺

wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Fatty Acid Soaps

The inventive composition may include soap. The term “soap” is used here in its popular sense, i.e., the alkali metal or alkanol ammonium salts of aliphatic alkane- or alkene monocarboxylic acids preferably having about 6 to 22 carbon atoms, more preferably about 6 to about 18 or about 12 to 18 carbon atoms. They may be further described as alkali metal carboxylates of aliphatic hydrocarbons. Sodium, potassium, mono-, di- and tri-ethanol ammonium cations, or combinations thereof, are suitable for purposes of this invention. In general, sodium soaps are used in the compositions of this invention, but from about 1% to about 25% of the soap may be potassium soaps. The soaps may contain unsaturation in accordance with commercially acceptable standards. Excessive unsaturation is normally avoided to minimize color and odor issues.

Soaps may be made by the classic kettle boiling process or modern continuous soap manufacturing processes wherein natural fats and oils such as tallow or coconut oil or their equivalents are saponified with an alkali metal hydroxide using procedures well known to those skilled in the art. Alternatively, the soaps may be made by neutralizing fatty acids, such as lauric (C12), myristic (C14), palmitic (C16), or stearic (C18) acids with an alkali metal hydroxide or carbonate.

Amphoteric Surfactants

One or more amphoteric surfactants may be used in this invention. When used, the amphoteric surfactant(s) may preferably be present from about 1, 2 or 3% by wt. up to about 5, 6 or 7% by wt. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:
R¹—[—C(O)—NH(CH₂)ₙ—]ₘ—N⁺—(R²)(R³)X—Y
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula:
R¹—N⁺—(R²)(R³)CH₂CO₂⁻
and amido betaines of formula:
   R¹—CONH(CH₂)ₙ—N⁺—(R²)(R³)CH₂CO₂⁻
where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula:
R¹—N⁺—(R²)(R³)(CH₂)₃SO₃⁻
Or
R¹—CONH(CH₂)ₘ—N⁺—(R²)(R³)(CH₂)₃SO₃⁻
where m is 2 or 3, or variants of these in which —(CH₂)₃SO₃⁻is replaced by
   —CH₂C(OH)(H)CH₂SO₃⁻

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the cleansing composition of the present invention. When used, the nonionic surfactant(s) may preferably be present from about 5, 10 or 15% by wt. up to about 25, 35 or 40% by wt.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Structurants

The inventive composition preferably includes one or more structurants to increase viscosity at zero shear and/or increase the ability of the composition to suspend particles. Advantageously the structurants are present in a total concentration of at least 0.1% by wt. Preferably the structurants are present in a total concentration of at least about 10, 20 or 25% by wt. and less than about 50, 40 or 30% by wt.

Useful structurant(s) of the invention can be water soluble, water dispersible or water insoluble. Water soluble structurants preferably include water soluble polymers and electrolytes.

Suitable water soluble polymers include polyalkylene oxides such as polyethylene glycols (PEG's), polypropylene glycols and polypropylene glycol oleates; cross-linked polyacrylates such as Carbopol (™) (polymers available from Goodrich); acrylates and copolymers thereof, polyvinylpyrrolidone and copolymers thereof; polyethylene imines; sucrose esters; and mixtures thereof, natural gums including alginates, guar, xanthan and polysaccharide derivatives including carboxy methyl cellulose, ethyl methylcellulose and hydroxyethylcellulose, hydroxyethyl methylcellulose and hydroxypropyl guar; and mixtures thereof, and the like. Other preferred water-soluble resins include polyvinyl alcohol, polyacrylamide, polyvinyl methyl ether-maleic anhydride, polymaleic anhydride, styrene maleic anhydride, polyacrylic acid salts, acrylamide copolymers, casein, and ethylene-maleic anhydride resin and the like.

Suitable electrolytes include alkali and alkaline earth salts such as halides, ammonium salts and sulphates, and the like. Suitable dispersible inorganic materials include clays such as synthetic hectorite (laponite), kaolin, bentonite or other clay derivatives or blends thereof. Other useful materials include amorphous silica, corn starch and bentonite talc. Clay(s) may be used in conjunction with an electrolyte salt capable of causing the clay to thicken.

Water insoluble structurants preferably have a melting point in the range 40°-100° C., more preferably at least 50° C., notably 50° C. to 90° C. Suitable materials include fatty acids and derivatives thereof, particularly those having a carbon chain of 12 to 24 carbon atoms. Examples are lauric, myristic, palmitic, stearic, hydroxystearic, arachidic and behenic acids, fatty esters of mono, di or tri glycerides; fatty acid monoglyceride polyglycol ethers, glycerol tallowates and mixtures thereof and the like. Useful sources of these aforementioned fatty acids are coconut, topped coconut, palm, palm kernel, babassu and tallow fatty acids and partially or fully hardened fatty acids or distilled fatty acids. Other suitable water insoluble structurants include alkanols of 8 to 20 carbon atoms, particularly cetyl alcohol and sugar fatty esters or fatty esters of dextrin and the like. These materials generally have a water solubility of less than 5 g/litre at 20° C.

Further examples of structurants are given in the International Cosmetic Ingredient Dictionary, Fifth Edition, 1993, published by CTFA (The Cosmetic, Toiletry & Fragrance Association), incorporated herein by reference.

Cationic Skin Conditioning Agents

An optional component in compositions according to the invention is a cationic skin feel agent or polymer. When used, the cationic skin feel agent or polymer may preferably be present from about 0.1, 0.3 or 0.5% by wt. to about 1, 1.5 or 2% by wt.

Suitable examples include cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Cationic Surfactants

One or more cationic surfactants may also be used in the inventive cleansing composition. When used, the cationic surfactant(s) may preferably be present from about 1, 5 or 10% by wt. up to about 11, 12 or 15% by wt.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued Mar., 27, 1973; and “Surface Active Agents and Detergents” (Vol. I & II) by Schwartz, Perry & Berch, both of which are also incorporated into the subject application by reference.

Non-Propellant Solubilizing Agent(s)

An effective concentration of liquid or flowable non-propellant solubilizing agent(s) are required for the present invention to solubilize and/or disperse the surfactant(s) with the other components of the cleansing composition to form a uniform blend prior to dispensing. Non-propellant solubilizing agent(s) may be selected from any compound that can solubilize or disperse the surfactants in the composition and that is/are sufficiently volatile to substantially evaporate after dispensing the inventive composition from an aerosol container. Advantageously at least about 5, 10 or 15% by wt. of the solubilizing agents will evaporate upon dispensing at 25 C at approx. 760 torr or 101 Kpa, preferably after about 10 seconds time. Suitable solubilizing agents include ethanol, water, methyl C6 to C26 esters (e.g. methyl lactate, metheyl soyate, and the like), acetone, heptane, diethylene alcohol, dimethyl ether, dipropylene glycol, ethanolamine, ethyl acetate, hexanes, isobutanol, isopentyl acetate, methanol, propanol and the like.

Useful solubilizing agents may also include surfactants that are liquid or flowable at processing temperatures for the inventive composition capable of dissolving or dispersing other surfactants. Preferably the total solubilizing agents are present at about 20, 30 or 40% by wt. to about 60, 70 or 80% by wt. More preferably the ratio of total surfactant(s) to total non-propellant, non-surfactant solubilizing agent(s) is in the range of 3:1 to 1:30.

Although not wishing to be bound by the following theory, it is believed that such solubilizing agent(s) enable a substantial portion of the surfactant base to blend intimately with the propellant(s) whereupon a substantially uniform, self-supporting solid is dispensed leaving a porous network upon substantial evaporation of the propellant(s) and optionally a substantial amount of the solubilizing agent(s). Useful solubilizing agent(s) having a boiling point range of about 26 to 104° C. at approx. 760 torr or 101 Kpa. Preferably a substantial portion, e.g. greater than about 10, 20 or 30% by wt. of the solubilizing agents are volatile, e.g. having boiling points of less than about 85, 75 or 70 C at approx. 760 torr or 101 Kpa. Water may advantageously be used as a solubilizing agent alone or in combination with other agents, preferably in a concentration range of about 10, 20 or 30 to 40, 50 or 60% by wt. of the total solubilizing agents.

Heat Producing Compositions

Heat-producing cosmetic compositions produce a very pleasant sensation and are advantageously used in a preferred embodiment of the invention. Useful systems for generating warmth have been described. For example, EP-A-27730 describes a cosmetic composition which may for example be a hair treatment or hand treatment composition and which generates heat on contact with water. In this application, heat generation is alleged to be provided by the presence of polyethylene oxides, polypropylene oxides, and derivatives thereof, and a 2 to 9 carbon atom alkaline glycol. EP-A-586929 describes a two pack system which generates heat on the addition of water, and produces a physiologically compatible salt that generates heat on mixture with water. Also contributing to the heat felt by the user on hydration of the product is the heat of hydration of polyethylene glycol. GB-A-1357000 describes a topical cosmetic composition comprising an anhydrous polyol and an absorbent particulate filler material. Suitable polyols are said to include propylene glycol, glycerol, 1,3 butylene glycol, and polyethylene glycols having an average molecular weight of from 2 to 300 and from 1000 to 6000. U.S. Pat. No. 6,287,580 discloses a self-warming cosmetic composition which delivers skin conditioning agents and is based upon a redox system of iron powder and a catalyst such as charcoal to provide the warmth. The system is activated with moisture and air. The self-warming composition is comprised of: (i) from about 0.1 to about 30% by weight of silicone oil or carboxylic ester as a skin conditioning agent; and (ii) from about 1 to about 95% by weight of a redox system based upon iron powder and a high surface area catalyst which is charcoal.

U.S. Pat. No. 4,839,081 discloses a two-stage chemically heated liquid soap composition dispensed as a cleansing composition for the face and hands derived from a novel double reductant and single oxidant redox system which includes hydrogen peroxide and a combination of sodium sulfite and ascorbic acid with a suitable catalyst. U.S. Pat. No. 4,439,416 discloses a cosmetic preparation which is effective for providing a cosmetic product that is heated at time of use by the heat of an exothermic reaction, and a process for carrying out such heating. The self-heating cosmetic preparation is composed of at least two discrete parts particularly suitable for providing a heated cosmetic product at time of use has one part comprising a hydride and a second part comprising a reducible material adapted to react exothermically with said hydride on contact therewith, and a cosmetic base in which one of said parts is incorporated, whereby at time of use the parts combine and I interact exothermically to issue as self-heated cosmetic preparation. U.S. Pat. No. 4,110,426 describes nonaqueous compositions, especially cosmetic preparations such as shaving creams which are rendered self-heating by including iherein a compound containing at least one boron-oxygen-boron linkage, such as triethoxyboroxine, which reacts exothermically with water or other protic material. U.S. Pat. No. 4,088,751 discloses a packaged self-heating cosmetic, such as a shaving cream, hand lotion, depilatory, facial, or a shampoo, including separate exothermically reactive reductant material, such as 2-thio-4-oxypyrimidine or thiohydantoin or derivatives thereof, and an oxidant, which reacts with the reductant, generating heat. The reducing agent employed is preferably 2-thio-4-oxypyrimidine or its tautomer, 2-mercapto-4-hydroxypyrimidine, or alkyl-substituted derivative thereof, and the oxidizing agent is preferably aqueous hydrogen peroxide.

U.S. Pat. No. 3,250,680 relates to the use of finely divided solid adsorbent materials which are capable of exothermically reacting with water. Illustrative of these materials are silica gel, activated alumina and synthetic zeolites. U.S. Pat. No. 4,626,550 relates to similar heating systems with improved versions of zeolite based on the presence of potassium ions as replacement for some of the sodium ions. WO 93/08793 describes other exothermic agents which are reactive with water. The agents include kaolin, Fuller's Earth, china clay and bentonite. U.S. Pat. No. 6,455,058 describes a composition and method for scalp treatment. The hair and scalp treatment contains an anti-dandruff agent such as salicylic acid, polyethylenimine (PEI) and an amphoteric surfactant. The composition is incorporated into either an aqueous or anhydrous solvent system. The anhydrous formulas have self-heating action whenever they are applied on wet hair. U.S. Pat. No. 6,309,655 discloses a cosmetic composition which combines a warming effect contributed by zeolite being exposed to water during the skin cleansing process. In addition to the commonly used zeolite, other heat-generating ingredients have been employed in a variety of compositions. U. S. Pat. No. 3,341,418 discloses a shaving composition wherein heat is generated by an oxidation-reduction reaction, which occurs when an oxidant, such as hydrogen peroxide or urea hydrogen peroxide from one compartment of the dispensing unit is mixed with reductants from a second compartment, which includes thiourea and a compound such as 1-phenyl-2-thio barbituric acid. U.S. Pat. No. 3,702,302 also employs the heat generation from an oxidation-reduction reaction using potassium sulfate as the reductant component. Similarly, U.S. Pat. No. 4,088,751 discloses a cosmetic composition that also uses specific reductants to effect heat generation from an oxidation-reduction reaction. U.S. Pat. No. 4,110,426 discloses a heat generating cosmetic composition in a surfactant carrier formulation, wherein compounds having at least one boron-oxygen-born linkage react exothermically with water or other protic materials to release heat. U.S. Pat. No. 6,540,989 discloses a heat generating rinse-out hair conditioner that relies on glycol as the warming agent. The warming effect is achieved when the glycol comes into contact with water.

Any suitable heat generating exothermic salt(s), compound(s) or blends thereof can be used that are compatible with the cleansing composition of the invention and that are safe to use on skin. The salt(s) and/or compound(s) can be incorporated into the substantially anhydrous base at any amount that results in a product that provides the desired level of heat and is aesthetically acceptable for its intended use. Substantially anhydrous is herein defined as less than 15, 10, 5, 3, 2, 1, or 0.5 wt. % water. The total amounts of exothermic salt(s) and/or compound(s) that can be included in a typical composition can range from 2, 5, 7 or 10 wt. % to 80 wt. % and preferably from 20 wt. % to 70 wt. % and more preferably between 20 to 60 wt. % depending upon the exothermic salt or compound selected and degree of heat required from the composition.

Optional Ingredients

The compositions may further comprise antimicrobials such as 2-hydroxy-4,2′,4′trichlorodiphenylether (DP300); preservatives such as dimethyloldimethylhydantoin (Glydant XL1000), parabens, sorbic acid etc., and the like.

Antioxidants such as, for example, butylated hydroxytoluene (BHT) and the like may be used advantageously in amounts of about 0.01% or higher if appropriate.

Skin Conditioning Agents

Skin conditioning agents may be advantageously used in the present invention. A skin conditioning agent, also known as an emollient is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

The skin conditioning agent(s) or emollient(s) may consist of a single component or may be a mixture of two or more components. In addition, the emollient itself may act as a carrier for other components one may wish to add to the foaming cleansing composition. Emollients are preferably present in a concentration greater than about 1, 2 or 5% by weight to a maximum of about 10, 15 or 20% by weight.

Useful examples of hydrophillic emollients (also known as humectants) include polyhydric alcohols, e.g. glycerine and propylene glycol, and the like; polyols such as the polyethylene glycols listed below and the like; saccharide(s) and/or polysaccharide(s) such as sucrose, sorbitol; and urea derivatives such as hydroxyethyl urea and the like may be advantageously used as a skin conditioning agent as well as a structurant.
Polyox WSR-205 PEG 14M,
Polyox WSR-N-60K PEG 45M, or
Polyox WSR-N-750 PEG 7M.

Hydrophillic emollients may also include C3 to C6 diol(s) or triol(s) or blends thereof. Other specific useful examples of hydrophillic emollients include any of the following or blends thereof: alcaligenes polysaccharides; algae extract; aloe barbadensis leaf extract; bacillus/rice bran extract/soybean extract ferment filtrate; black strap powder; diglycereth-7 malate; diglycerin; diglycol guanidine succinate; erythritol; fructose; glucose; glucoronolactone; glycereth-7 glycolate; glycerin; glyceryl dimaltodextrin; glycol; hesperetin laurate; 1,2,6-hexanetriol; honey; hydrogenated honey; hydrogenated starch hydrolysate; hydrolyzed wheat protein/PEG-20 acetate copolymer; hydroxypropyltrimonium hyaluronate; inositol; lactic acid; lacitol; maltitol; maltose; mannitol; mannose; methoxy PEG-7; methoxy PEG-10; methoxy PEG-16; methoxy PEG-25; methoxy PEG-40; methoxy PEG-100; PEG 4; PEG-6; PEG-7; PEG-8; PEG-9; PEG-10; PEG-12; PEG-14; PEG-16; PEG-18; PEG-20; PEG-32; PEG40; PEG-45; PEG-55; PEG-60; PEG-75; PEG-90; PEG-75; PEG-90; PEG-100; PEG-135; PEG-150; PEG-180; PEG-200; PEG-220; PEG-240; PEG-800; PEG-15 butanediol; PEG-3-methyl ether; PEG-4 methyl ether; PEG-5 pentaerythrityl ether; polyglyceryl sorbitol; potassium dextrin octenylsuccinate; potassium PCA; PPG-6 sorbeth-245; PPG-6 sorbeth-500; propylene glycol; rosa canina seed extract; sodium acetylated hyaluronate; sodium dextrin octenylsuccinate; sodium glucuronate; sodium PCA; sorbeth-6; sorbeth-20; sorbeth-30; sorbeth-40; sorbitol; sorbityl silanediol; sucrose; TEA dextrin octenylsuccinate; trehalose; triglycereth-7 citrate; trioxaundecanedioic acid; tripropylene glycol; urea; urea-d-glucuronic acid; xylitol; xylose and the like.

Useful hydrophobic emollients (some of which may also be used as structurants) include the following:

(a) silicone oils and modifications thereof such as linear and cyclic polydimethylsiloxanes; amino, alkyl, alkylaryl, and aryl silicone oils;

(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;

(c) waxes such as carnauba, spermaceti, beeswax, lanolin, and derivatives thereof;

(c) hydrophobic plant extracts;

(e) hydrocarbons such as liquid paraffins, vaseline, microcrystalline wax, ceresin, squalene, pristan and mineral oil;

(f) higher fatty acids such as lauric, myristic, palmitic, stearic, behenic, oleic, linoleic, linolenic, lanolic, isostearic, arachidonic and poly unsaturated fatty acids (PUFA);

(g) higher alcohols such as lauryl, cetyl, stearyl, oleyl, behenyl, cholesterol and 2-hexydecanol alcohol;

(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;

(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; and

(o) mixtures of any of the foregoing components, and the like.

Dispensing System and Propellants:

Suitable aerosol container dispensing systems may be preferably used to dispense the inventive compositions. These systems include lined aluminum standard aerosol cans (available from suppliers such as CCL Container (Ontario, Canada) or Exal (Buenos Aires, Argentina)) or lined 3 piece steel cans (available from suppliers such as United States Can Corp (Lombard, Ill.) or Crown Cork and Seal (Philadelphia, Pa.)).

Dispensing systems according to the invention include propellant(s). Propellant(s) are defined herein as any component or combination of components that exist as a gas at room temperature (approx. 23° C.) and ambient pressure (approx. 760 torr or 101 Kpa) that is (are) capable of causing the dispensing of the inventive composition from its container. Suitable propellant(s) include any liquefiable gas that may be used for aerosol containers. Examples of suitable propellants include dimethyl ether, propane, n-butane and isobutane, and hydrofluorocarbons such as propellant 152A, propellant 17A, used singly or admixed. Other examples of suitable propellants include nitrogen, carbon dioxide and compressed air. Preferably one or more liquifiable volatile hydrocarbons, such as isobutane or blends thereof, optionally containing dimethyl ether are used. The propellant is present in the packaged composition at any required and suitable level effective to dispense the inventive composition, but is preferably present in the container at levels sufficient to produce a satisfactory dispensing pressure, which will typically be 55±30 psig, preferably 55±20 psig at 23° C.

Optional Active Agents

Advantageously, active agents other than skin conditioning agents such as emollients as defined above may be added to the cleansing composition in a safe and effective amount during formulation to treat the skin during the use of the product. These active ingredients may be advantageously selected from antimicrobial and antifungal actives, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof; and the like.

Suitable active agents may be selected from water soluble or hydrophilic active agents, oil soluble active. or hydrophobic agents, pharmaceutically-acceptable salts and mixtures thereof. Advantageously the agents will be soluble or dispersible in the cleansing composition. Hydrophobic agents if present are advantageously at maximum levels of about 5, 4, 3, 2, 1, 0.5 or 0.1% by wt. The term “active agent” as used herein, means personal care actives which can be used to deliver a benefit to the skin and/or hair and which generally are not used to confer a conditioning benefit, as is conferred by humectants and emollients previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the composition of the present invention comprise from about 0.01% to about 50%, more preferably from about 0.05% to about 25%, even more preferably 0.1% to about 10%, and most preferably 0.1% % to about 5%, by weight of the active agent component.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Patent Specification No. 556,957; ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Barnet Products Distributor), and the like.

All publications, patents, and patent applications cited herein are incorporated herein by reference in their entirety. Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated. Physical test methods are described below:

### EXAMPLE 1

### Inventive Cleansing Compositions 1-14 Were Prepared as Shown in Table 1 Using the Procedure Described Below

TABLE 1
% BY WT.

| Component | 1 | 2 | 3 | 4 | 5 | 6 | 7 | 8 | 9 | 10 | 11 | 12 | 13 | 11 | 12 | 13 | 14 |
|---|---|---|---|---|---|---|---|---|---|---|---|---|---|---|---|---|---|
| Ethanol | 27 | 22 | 26 | 26 | 23.5 | 32 | 33 | 31 | 28 | 45 | 20 | 25 | 22.5 | 45 | 20 | 25 | 22.5 |
| Syndet noodles | 30 | 47 | 40 | 40 | 43 | 43 | 38 | 26 | 28 | 27 | 27 | 32.5 | 30 | 30 | 32 | 33 | 33 |
| (1) | | | | | | | | | | | | | | | | | |
| Water | Qs | Qs | Qs | Qs | Qs | Qs | Qs | Qs | Qs | Qs | Qs | Qs | Qs | Qs | Qs | Qs | Qs |
| | to | to | to | to | to | to | to | to | to | to | to | to | to | to | to | to | to |
| | 100 | 100 | 100 | 100 | 100 | 100 | 100 | 100 | 100 | 100 | 100 | 100 | 100 | 100 | 100 | 100 | 100 |
| Sodium C14-16 | 0 | 0 | 4.5 | 0 | 0 | 0 | 0 | 4 | 3 | 2 | 3 | 3 | 2.5 | 3 | 3 | 3 | 3 |
| Olefin | | | | | | | | | | | | | | | | | |
| Sulfonate | | | | | | | | | | | | | | | | | |
| Ammonium | 2.35 | 1.03 | 0 | 0 | 0 | 0 | 0 | 0 | 0 | 0 | 0 | 0 | 0 | 0 | 0 | 0 | 0 |
| Laureth Sulfate | | | | | | | | | | | | | | | | | |
| PEG-5 | 0.25 | 0.23 | 0 | 0 | 0 | 0 | 0 | 0 | 0 | 0 | 0 | 0 | 0 | 0 | 0 | 0 | 0 |
| Cocamide | | | | | | | | | | | | | | | | | |
| Cocamide MEA | 0.5 | 0.02 | 0 | 0 | 0 | 0 | 0 | 0 | 0 | 0 | 0 | 0 | 0 | 0 | 0 | 0 | 0 |
| Ammonium | 2.9 | 1.3 | 0 | 0 | 0 | 0 | 0 | 0 | 0 | 0 | 0 | 0 | 0 | 0 | 0 | 0 | 0 |
| Lauryl Sulfate | | | | | | | | | | | | | | | | | |
| Sodium | 0 | 0 | 0 | 0 | 4 | 4 | 3 | 0 | 0 | 0 | 0 | 0 | 0 | 0 | 0 | 0 | 0 |
| tetradecyl | | | | | | | | | | | | | | | | | |
| sulphate | | | | | | | | | | | | | | | | | |
| Cocoamidopropyl | 0 | 0 | 0 | 4.5 | 0 | 0 | 0 | 0 | 0 | 0 | 0 | 0 | 0 | 0 | 0 | 0 | 0 |
| betaine | | | | | | | | | | | | | | | | | |
| Polyvinyl- | 0.5 | 0.4 | 0.5 | 0.5 | 0.5 | 0.5 | 0 | 0 | 0 | 0 | 0.5 | 0.5 | 0.5 | 0 | 0.5 | 0.5 | 0.5 |
| pyrrolidone K30 | | | | | | | | | | | | | | | | | |
| (2), (3) | | | | | | | | | | | | | | | | | |
| Cetyl Alcohol | 0 | 0 | 0 | 0 | 0 | 0 | 0 | 0 | 5 | 5 | 0 | 0 | 0 | 0 | 0 | 0 | 0 |
| Stearyl Alcohol | 0 | 0 | 0 | 0 | 0 | 0 | 0 | 0 | 0 | 0 | 0 | 0 | 0 | 2 | 0 | 0 | 0 |
| PEG 400 (4, 5) | 0 | 0 | 0 | 0 | 0 | 0 | 4 | 1 | 1 | 1 | 0 | 0 | 0 | 3 | 1.5 | 1.5 | 2 |
| Fragrance | 1 | 1 | 1 | 1 | 1 | 1 | 1 | 1 | 1 | 1 | 1 | 1 | 1 | 1 | 1 | 1 | 1 |
| Propellant A31 | 6 | 0 | 0 | 6 | 0 | 0 | 0 | 0 | 0 | 0 | 0 | 0 | 0 | 0 | 0 | 0 | 0 |
| (6) | | | | | | | | | | | | | | | | | |
| Propellant A50 | 0 | 6 | 6 | 0 | 6 | 6 | 6 | 6 | 6 | 6 | 6 | 6 | 6 | 6 | 6 | 6 | 6 |
| (7) | | | | | | | | | | | | | | | | | |

Notes:
(1) Noodle composition is shown in Table 2 as blend 1; other blends in Table 2 may be suitably used.
(2) Obtained from BASF Company (Ledgewood, NJ)
(3) ISP Co. (Wayne NJ)
(4) Laboratoires Serobiologiques- Division of Cognis France (Pulnoy, France)
(5) Clariant Corporation (Mount Holly NC)
(6) Composition of propellant A31 is C4H10 Isobutane
(7) Composition of propellant A50 is Isobutane-Butane Mix

Sample Preparation Method:

Set up a mixing bowl in a hot water bath, kept steady at about 100-120 F (37.8 C-48.9 C), Add in solvents, low Krafft point surfactants (liquids), and dissolvable structurants with light mixing (z-blade, propeller mixer, etc. at ˜100 rpm)
Once a homogenous solution is prepared add in high Krafft point surfactants (soap noodles, etc) and monitor heat (add slight excess of noodles to be sure solubility limit is reached.
Heat to (and hold temperature at) 90-95 F (32.2 C-35 C), adjusting stirring rate as needed (vigorous without splashing) until solution is homogenous.

Once a fluid consistency is obtained with minimal to no solid residue, quickly filter through 40-50 mesh screen, and bottle in aerosol container: crimped lid and pressurized with propellant.

TABLE 2
Composition of Syndet Noodles Blends

| | Blends | | | | |
|---|---|---|---|---|---|
| Component | 1 | 2 | 3 | 4 | 5 |
| Sodium Cocoyl | 52 | 45 | 51.5 | 54.5 | 49 |
| Isethionate | | | | | |
| Coconut Fatty Acid | 6 | 8 | 9.7 | 6 | 7 |
| Sodium Chloride | 0.5 | 1 | 0.8 | 0.5 | 0.7 |
| Stearic Acid | 15 | 18.5 | 20 | 16 | 21 |
| Sodium Cocoate | 6 | 10 | 5 | 6 | 8 |
| Preservative | 1 | 1 | 1 | 1 | 1 |
| Titanium Dioxide | 0.5 | 0.5 | 0.5 | 0.5 | 0.5 |
| Cosurfactant | 3 | 2 | 2 | 3 | 2 |
| (cocamidopropyl | | | | | |
| betaine) | | | | | |
| Sodium Isethionate | 10 | 8 | 5 | 7.5 | 5.3 |
| Water | qs to | qs to | qs to | qs to | qs to |
| | 100 | 100 | 100 | 100 | 100 |

Method of Preparing Syndet Noodles:

Stearic acid is melted in a sigma or equivalent mixer. Approx. 25% solution of sodium hydroxide is used for in-situ generation of sodium stearate. Enough time is provided to dissolve sodium stearate by heating the mass to about 120 C with continuous mixing. Once sodium stearate is dissolved then the acyl isethionate such as sodium cocoyl isethionate is added and mixed. After that the rest of the ingredients are added and the moisture is reduced to about 5% by wt. via conventional dehydration techniques. The blend is then cooled and solidified in a spray dryer or a chill roll to form the noodles.

Methods

a) Dispensed Volume Stability Method

Dispense 100 mL (“Instant Dispensed Volume”) of product into a graduated volumetric flask or graduated cylinder at approx. 23 C making sure that no air is entrained in the flask or cylinder.
T₀ is the time that the product is dispensed to the desired mark on the flask or cylinder.
Note the volume of product after desired intervals of time from T₀—e.g. at 2 hour intervals.
   b) Lather Volume Determination Method

Lather volume is measured by using the following water volumetric displacement method.
1. Wash hands and lather thoroughly with water and a sodium palmitate and/or sodium cocoate soap (such as Ivory® available from Procter & Gamble) for 30 seconds at about 30 C, rinse hands for 30 seconds. Do not dry hands.
2. Dispense about 5 grams of aerosol sample onto one hand.
3. Add 1 mL of water at about 30 C to hand with product. Circulate opposite hand over hand with product and lather for 10 seconds at 1 circular stroke per second. Add another 1 mL of water onto initial hand with product, and then circulate opposite hand over the hand with the product for 30 seconds at a rate of 1 circular stroke per second.
4. Dip both the hands in water under an inverted funnel having an attached graduated cylinder. The generated lather is collected by the inverted funnel and quantified via the attached graduated cylinder. Lather volume is measured in mls at about 30 C.
   Results:

The inventive product is seen to produce copious amounts of creamy, persistent lather following the wash procedure of the Lather Volume Determination Method. Comparative aerosol products such as mousses, shave gels, hair sprays, etc. are expected to be relatively devoid of lather after the completion of the above wash procedure.

While not wishing to be bound by the following theory, this is believed due to the relative instability of the lathering (surfactant system) in such comparative products that are typically dispensed already as lather.

c) Lather Coefficient Determination Method

As discussed above, lather is defined as a foamed structure consisting of and necessitating the presence of both bubbles and cream or liquid. A Lathering Coefficient is defined according to the following 1-6 scale using the Lather Volume Determination Method to determine lather volume.
1—having no visible or measurable lather (foam and bubbles)
2—having visible lather but below about 1 to 5 mLs in volume.
3—having visible lather greater than 5 mL but less than or equal to 50 mL in volume.
4—having visible lather greater than 50 mL but less than or equal to 100 mL in volume.
5—having visible lather greater than 100 mL but less than or equal to 150 mL in volume.
6—having visible lather greater than 150 mL in volume.
   d) Krafft Point Determination

Make up a 10% by wt. solution of surfactant or other sample in water. If needed, heat the system to dissolve the sample completely. Transfer the clear solution to a glass test tube. Place the test tube in a beaker equipped with a stirrer and filled with sufficient water to evenly cool the surfactant or sample solution. The solution should be cooled with continuous stirring and the temperature should be continuously recorded. Note the temperature when the crystallization process begins such that the solution becomes turbid. This temperature is taken as the Krafft point. If the crystallization temperature is below room temperature, add ice to the beaker to cool the test tube below room temperature to measure the subambient Krafft point.

e) Foam Specific Gravity Determination Method

Foam specific gravity may be measured using a stainless steel pycnometer. First, the pycnometer is rinsed with tap water followed by distilled water. Than it is rinsed with acetone, dried, and allowed to come to room temperature (approx. 23 C). The empty pycnometer is tare weighed. The body of the pycnometer is carefully filled with water, avoiding air bubbles. The cover is placed on and collar screwed into place. Excess water is wiped away and the filled pycnometer is weighed.

After cleaning and drying, the product for which the specific gravity is to be measured is added to the pycnometer and the cover is carefully pressed down until seated. Excess sample is expelled through the center is wiped away and the collar is screwed on. Excess sample is cleaned from the outside of the pycnometer and the filled pycnometer is weighed. Specific gravity at 23 C is calculated using the following equation:
g sample/g H₂O.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

We claim:

1. An aerosol cleansing composition that is self-supporting after dispensing comprising:
a. surfactant(s) with a Krafft point greater than 27 C in a total concentration range of about 20 to 60% by wt.;
b. optionally surfactant(s) with a Krafft point less than or equal to 27 C in a total concentration range of up to about 25% by wt.;
c. non-propellant solubilizing agent(s) in a total concentration range of about 20 to 80% by wt.;
d. propellant(s) in a total concentration range of about 2 to 20% by wt.; and
e. wherein the dispensed composition retains about 90% of its dispensed volume after 24 hours (at approx. 23 C, 760 torr or 101 Kpa, and 70% RH).

2. The composition of claim 1 further comprising a lather coefficient of at least 4 (at approx. 23 C, 760 torr or 101 Kpa, and 70% RH).

3. The composition of claim 1 further comprising one or more structurants in a total concentration of at least 0.1% by wt.

4. The composition of claim 3 where the ratio of total structurants to total surfactants is in the range of about 0.2 to 5.

5. The composition of claim 1 further comprising total skin conditioning agent(s) in the concentration range of about 0.1 to 5% by wt.

6. The composition of claim 1 wherein the propellants are selected from hydrocarbons, dimethyl ether, carbon dioxide, nitrous oxide, compressed air, nitrogen, fluorohydrocarbons, or blends thereof.

7. The composition of claim 1 wherein the specific gravity of the cleansing composition measured 5 seconds after dispensing is at least about 0.25 (at approx. 23 C, 760 torr or 101 Kpa, and 70% RH).

8. The composition of claim 1 further comprising soap in a concentration of less than about 40% by wt.

9. The composition of claim 1 further comprising one or more skin active agent(s) in the total concentration range of about 0.02 to 10% by wt.

10. The composition of claim 9 wherein the one or more skin active agent(s) are selected from antibacterial agent(s), anti-aging agent(s), anti-acne agent(s) or skin firming agent(s).

11. The composition of claim 1 further comprising less than about 90% by wt. of water.

12. The composition of claim 1 wherein the composition is substantially anhydrous.

13. The composition of claim 12 further comprising an effective concentration of heat generating compound(s) sufficient for generating an increase in temperature of the composition with use.

14. A packaged cleansing product comprising:
a. a container having a valve;
b. an aerosol cleansing composition that is self-supporting after dispensing including:
1. surfactant(s) with a Krafft point greater than 27 C in a total concentration range of about 20 to 60% by wt.;
2. optionally surfactant(s) with a Krafft point less than or equal to 27 C in a total concentration range of up to about 25% by wt.;
3. non-propellant solubilizing agent(s) in a total concentration range of about 20 to 80% by wt.;
4. propellant(s) in a total concentration range of about 2 to 20% by wt.; and
c. wherein the dispensed composition retains about 90% of its dispensed volume after 24 hours (at approx. 23 C, 760 torr or 101 Kpa, and 70% RH). and
d. wherein the valve is capable of releasing the cleansing composition when the internal pressure exerted by the cleansing composition is greater than the outside air pressure.

15. A method for cleansing the skin without rinsing with water, comprising the steps of:
a. applying the self-supporting aerosol cleansing composition of claim 1 to the skin;
b. distributing the applied composition to the area of skin desired; and
c. wiping off the applied composition.
